(19) United States
(12) Patent Application Publication
(10) Pub. No.: US 2009/0299094 A1
(43) Pub. Date: Dec. 3, 2009

(54) PROCESS FOR PRODUCING ALPHA, BETA-UNSATURATED CARBOXYLIC ACID

(75) Inventors: Toshihiko Fukuda, Hiroshima (JP); Kazunori Matake, Hiroshima (JP); Atsushi Koizumi, Hiroshima (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Mitsubishi Rayon Co., Ltd., Tokyo (JP)

(21) Appl. No.: 11/917,422

(22) PCT Filed: Jun. 12, 2006
(86) PCT No.: PCT/JP2006/311710

§ 371 Date: Dec. 13, 2007

(30) Foreign Application Priority Data
Jun. 13, 2005 (JP) 2005-171890

(51) Int. Cl. C07C 57/03

(52) U.S. Cl. 562/598

(57) ABSTRACT

Disclosed is a method for producing an α,β-unsaturated carboxylic acid by oxidizing an olefin or an α,β-unsaturated aldehyde in a liquid phase in the presence of a noble metal catalyst, which can ensure operational safety at the time of stopping the reaction and prevent deterioration of the noble metal catalyst. The method for producing the α,β-unsaturated carboxylic acid by an oxidation reaction in which the olefin or the α,β-unsaturated aldehyde is oxidized in the liquid phase in the presence of the noble metal catalyst in a reactor, includes a step of stopping the oxidation reaction by supplying an inert gas to the reactor.

### TECHNICAL FIELD

The present invention relates to a method for producing an α,β-unsaturated carboxylic acid by carrying out a liquid-phase oxidation reaction.

### BACKGROUND ART

As a method for producing an α,β-unsaturated carboxylic acid, a method in which an olefin having 3 to 6 carbon atoms and oxygen are supplied to a reactor and the olefin is oxidized in a liquid phase in the presence of an activated palladium metal catalyst to obtain the α,β-unsaturated carboxylic acid is disclosed (Patent Document 1).

Patent Document 1: Japanese Patent Application Laid-Open No. Sho 60-155,148

### DISCLOSURE OF INVENTION

### Problem to be Solved by the Invention

In Patent Document 1, there is no description about a method for stopping a reaction, though there is a description of an activation method of a catalyst when the reaction is started. In the case of stopping the reaction by simultaneously stopping the supply of an olefin having 3 to 6 carbon atoms and oxygen for the purpose of inspection or repair of equipment, it is apprehended that a noble metal catalyst is oxidized and deteriorated by the dissolved oxygen in a liquid phase in a reactor after the reaction has been stopped. Further, it is apprehended that a gasified nonreacted olefin having 3 to 6 carbon atoms and oxygen may accumulate at an upper space portion of the reactor, and the dissolved oxygen in the reaction liquid may evaporate while a combustible gas is existing at the upper space portion of the reactor to raise oxygen concentration, which may cause explosion.

It is an object of the present invention to provide a method for producing an α,β-unsaturated carboxylic acid by oxidizing an olefin or an α,β-unsaturated aldehyde in a liquid phase in the presence of a noble metal catalyst, which can ensure operational safety at the time of stopping the reaction and prevent deterioration of the noble metal catalyst.

### Means for Solving the Problem

The gist of the present invention resides in a method for producing an α,β-unsaturated carboxylic acid by an oxidation reaction in which an olefin or an α,β-unsaturated aldehyde is oxidized in a liquid phase in the presence of a noble metal catalyst in a reactor to obtain the α,β-unsaturated carboxylic acid, the method comprising the step of stopping the oxidation reaction by supplying an inert gas to the reactor.

One preferable aspect of the present invention is the method for producing an α,β-unsaturated carboxylic acid, in which the oxidation reaction is carried out continuously by supplying the olefin or the α,β-unsaturated aldehyde, a solvent and molecular oxygen to the reactor, and supply of the oxygen is stopped before the inert gas is supplied in the step of stopping the oxidation reaction. In the step of stopping the oxidation reaction, supply of the olefin or the α,β-unsaturated aldehyde can also be stopped after the inert gas is supplied. A more preferable aspect of the present invention is the method for producing an α,β-unsaturated carboxylic acid, in which a rate of supply of the inert gas to be supplied to the reactor in the step of stopping the oxidation reaction is 1 to 100 times as much as a rate of supply of the molecular oxygen to be supplied in the oxidation reaction.

Another preferable aspect of the present invention is the method for producing an α,β-unsaturated carboxylic acid, in which a total volume of the inert gas to be supplied to the reactor at 0° C. and under 1 atm in the step of stopping the oxidation reaction is 1 to 1,000 times as much as a volume of a reaction liquid in the reactor.

Another preferable aspect of the present invention is the method for producing an α,β-unsaturated carboxylic acid, in which a total volume of the inert gas to be supplied to the reactor at 0° C. and under 1 atm in the step of stopping the oxidation reaction is 1 to 1,000 times as much as a volume of the reactor.

Another preferable aspect of the present invention is the method for producing an α,β-unsaturated carboxylic acid, in which a reducing agent is further supplied to the reactor in the step of stopping the oxidation reaction. For example, as the reducing agent, an olefin or an α,β-unsaturated aldehyde which is liquid at a temperature and a pressure in the reactor can be used. A more preferable aspect of the present invention is the method for producing an α,β-unsaturated carboxylic acid, in which an amount of the reducing agent (g) to be supplied to the reactor in the step of stopping the oxidation reaction is, based on a volume V (L) of the reaction liquid in the reactor, from V×100 to V×2,000.

### EFFECT OF THE INVENTION

According to the present invention, in a method for producing an α,β-unsaturated carboxylic acid by oxidizing an olefin or an α,β-unsaturated aldehyde in a liquid phase in the presence of a noble metal catalyst, operational safety at the time of stopping the reaction can be ensured and deterioration of the noble metal catalyst can be prevented.

### BEST MODE FOR CARRYING OUT THE INVENTION

Hereinafter, the present invention will be explained in detail.

In the present invention, an oxidation reaction in which an olefin or an α,β-unsaturated aldehyde which is a raw material is oxidized by molecular oxygen to produce an α,β-unsaturated carboxylic acid is carried out in a liquid phase in the presence of a noble metal catalyst. By such an oxidation reaction, the α,β-unsaturated carboxylic acid is produced in high selectivity and high yield. The oxidation reaction may be carried out in a continuous operation or a batchwise operation, however, the continuous operation is preferable in point of productivity.

As the olefin, the one having 3 to 6 carbon atoms is preferable, and, for example, propylene, isobutylene, 1-butene, or 2-butene can be listed. As the α,β-unsaturated aldehyde, for example, acrolein, methacrolein, crotonaldehyde (β-methylacrolein), or cynnamaldehyde (β-phenylacrolein) can be listed.

The α,β-unsaturated carboxylic acid to be produced is the one in which a methyl group in the olefin has changed into a carboxyl group in the case that the raw material is the olefin, and the one in which an aldehyde group of the α,β-unsaturated aldehyde has changed into a carboxyl group in the case that the raw material is the α,β-unsaturated aldehyde. Concretely, for example, acrylic acid can be obtained in the case that the raw material is propylene or acrolein, and methacrylic acid can be obtained in the case that the raw material is isobutylene or methacrolein.

As a source of the molecular oxygen, air is economical and hence preferable, and pure oxygen or a mixed gas of pure oxygen and air can also be used, and if necessary, a mixed gas obtained by diluting air or pure oxygen with nitrogen, carbon dioxide, or water vapor can also be used. It is preferable that the molecular oxygen be supplied in a pressurized state to a reactor such as autoclave.

The solvent to be used in the oxidation reaction is not particularly limited, and water; an alcohol such as t-butanol or cyclohexanol; a ketone such as acetone, methyl ethyl ketone, or methyl isobutyl ketone; an organic acid such as acetic acid, propionic acid, n-butylic acid, isobutylic acid, n-valeric acid, or isovaleric acid; an organic acid ester such as ethyl acetate or methyl propionate; or a hydrocarbon such as hexane, cyclohexane, or toluene can be used. Among them, an organic acid having 2 to 6 carbon atoms, a ketone having 3 to 6 carbon atoms, and t-butanol are preferable. The solvent can be used alone or as mixed solvents of two or more kinds.

The noble metal catalyst contains a noble metal which catalyzes the oxidation reaction. As the noble metal, for example, palladium, platinum, rhodium, ruthenium, iridium, gold, silver, or osmium can be used. Among them, palladium, platinum, rhodium, ruthenium, iridium, or gold is preferable, and palladium is particularly preferable. The noble metal may be used alone or in combination of two or more kinds.

The noble metal catalyst may contain any metal (nonnoble metal) other than the noble metal. As the nonnoble metal, bismuth or tellurium is preferable. The nonnoble metal may be used alone or in combination of two or more kinds. The rate of the nonnoble metal in the metals contained in the noble metal catalyst is preferably 50 atomic % or less from the viewpoint of catalyst activity.

The noble metal catalyst may be a nonsupported type or a supported type. As a carrier to be used in the case of the supported type, for example, activated carbon, carbon black, silica, alumina, magnesia, calcia, titania, or zirconia can be listed. Among them, activated carbon, silica, or alumina is preferable. The carrier may be used alone or in combination of two or more kinds. In the case of the supported type catalyst, loading ratio of the noble metal is preferably 0.1 to 40% by mass to the carrier before the noble metal is supported, and more preferably 1 to 30% by mass.

Further, it is preferable to cause a polymerization inhibitor to exist in a reaction liquid by about 1 to 10,000 ppm in order to prevent polymerization of raw materials or products. As the polymerization inhibitor, for example, a phenol compound such as hydroquinone or paramethoxyphenol; an amine compound such as N,N′-diisopropylparaphenylenediamine, N,N′-di-2-naphtylparaphenylenediamine, N-phenyl-N′-(1,3-dimethylbutyl)paraphenylenediamine, or phenothiazine; or an N-oxyl compound such as 4-hydroxy-2,2,6,6-tetramethylpiperidine-N-oxyl or 4-benzoyloxy-2,2,6,6-tetramethylpiperidine-N-oxyl can be listed. The polymerization inhibitor may be used alone or in combination of two or more kinds.

Conditions of the oxidation reaction are properly selected depending on a solvent and a raw material to be used, and preferable conditions will be explained in the following.

The liquid volume in the reactor (expressed as V (L)) is preferably 10 to 80% of the volume of the reactor. The reaction temperature is preferably 30 to 200° C., and more preferably 50 to 150° C. The reaction pressure is preferably 0 to 10 MPaG, and more preferably 2 to 7 MPaG. The amount of the noble metal catalyst to be used is preferably 0.1 to 50% by mass to the liquid in the reactor, more preferably 0.5 to 30% by mass, and furthermore preferably 1 to 15% by mass. The noble metal catalyst may be used in a suspended state in the reaction liquid or in a fixed bed.

When the oxidation reaction is carried out continuously, an olefin or an α,β-unsaturated aldehyde, a solvent, and molecular oxygen are continuously supplied. It is preferable that each component be supplied continuously in the following condition. The feed rate (g/h) of the olefin or the α,β-unsaturated aldehyde which is a raw material is preferably V×10 to V×500. The feed rate (g/h) of the solvent is preferably V×100 to V×2,000. The feed rate (g/h) of molecular oxygen is preferably V×100 to V×2,000. Further, feed rate per hour of molecular oxygen is preferably 0.1 to 20 moles to 1 mole of the olefin or the α,β-unsaturated aldehyde which is a raw material, and more preferably 0.1 to 5 moles.

Now, a preferable aspect of the preparation method of the noble metal catalyst will be shown below.

Firstly, a noble metal compound and a carrier are added to the solvent in a desired order or simultaneously to prepare a fluid dispersion in which the carrier is dispersed. Secondly, a reducing agent is added to the fluid dispersion to reduce the noble metal at the same time to load the noble metal on the carrier.

The noble metal compound to be used at the time of the catalyst preparation is not particularly limited, however, a compound which contains a noble metal atom in an oxidized state is preferable. For example, a chloride, an oxide, an acetate, a nitrate, a sulfate, a tetraamine complex, or acetylacetonato complex of the noble metal is preferable. Among them, a chloride, an acetate, or a nitrate of the noble metal is more preferable.

In the case of preparing the noble metal catalyst which contains a nonnoble metal, a noble metal compound and a nonnoble metal compound may be jointly used. For example, the nonnoble metal can be contained in the noble metal catalyst by dissolving the nonnoble metal compound in a solvent of a liquid phase when the noble metal compound is reduced in the liquid phase.

As the solvent to be used at the time of the catalyst preparation, water is preferable, however, depending on solubility of the noble metal compound and the reducing agent and on dispersibility of a carrier in the case of using the carrier, an organic solvent like an alcohol such as ethanol, 1-propanol, 2-propanol, n-butanol, or t-butanol; a ketone such as acetone, methyl ethyl ketone, methyl isobutyl ketone, or cyclohexanone; an organic acid such as acetic acid, n-valeric acid, or isovaleric acid; or a hydrocarbon such as heptane, hexane, or cyclohexane can be used alone or in combination of more than one kind.

The reducing agent to be used at the time of the catalyst preparation is not particularly limited, and as the reducing agent, for example, hydrazine, formaldehyde, sodium borohydride, hydrogen, formic acid, a formate, ethylene, propylene, 1-butene, 2-butene, isobutylene, 1,3-butadien, 1-heptene, 2-heptene, 1-hexene, 2-hexene, cyclohexene, allyl alcohol, methallyl alcohol, acrolein, or mathacrolein can be listed.

The reducing temperature is variable depending on the noble metal compound or the reducing agent to be used, however, it is preferably −5 to 150° C. and more preferably 15 to 80° C. The reducing time is preferably 0.1 to 4 hours, more preferably 0.25 to 3 hours, and furthermore preferably 0.5 to 2 hours.

It is preferable to wash the noble metal catalyst deposited by reduction with water, a solvent, or the like to remove an impurity such as a chloride, acetate group, nitrate group, or sulfate group originated from the noble metal compound.

It is preferable to carry out the oxidation reaction using the noble metal catalyst thus obtained.

In the present invention, the oxidation reaction is stopped by supplying an inert gas to the reactor after the above-mentioned oxidation reaction is carried out (a step of stopping the oxidation reaction). In the case that the oxidation reaction is carried out continuously, it is preferable that supply of molecular oxygen be stopped before the inert gas is supplied in the step of stopping the oxidation reaction. It is possible to avoid a danger of explosion caused by an increase in an oxygen concentration in a gas at the upper space portion of the reactor and to prevent deterioration of the noble metal catalyst with molecular oxygen by supplying the inert gas to the reactor to purge molecular oxygen inside the reactor to outside the reactor at the time of stopping the oxidation reaction as mentioned above.

As the inert gas, nitrogen, carbon dioxide, or a rare gas such as helium, neon, or argon can be listed.

The position at which the inert gas is supplied to the reactor is not particularly limited, however, it is preferable to supply the inert gas to the liquid phase portion inside the reactor in order to purge molecular oxygen existing around the noble metal catalyst to outside the reactor more effectively.

The total volume of the inert gas to be supplied to the reactor, at 0° C. and under 1 atm, is preferably 1 to 1,000 times as much as a volume of a reaction liquid in the reactor and more preferably 2 to 100 times. Further, the total volume of the inert gas to be supplied to the reactor, at 0° C. and under 1 atm, is preferably 1 to 1,000 times as much as a volume of the reactor and more preferably 2 to 100 times. Consequently, oxygen concentration inside the reactor including the reaction liquid and the upper space portion of the reactor can be lowered, and hence the prevention of the deterioration of the noble metal catalyst by oxidation and the avoidance of the danger of explosion can be attained at the same time.

Further, in the case that the oxidation reaction is carried out continuously, the rate of supply of the inert gas to be supplied to the reactor is preferably 1 to 100 times as much as the rate of supply of molecular oxygen, and more preferably 1 to 10 times because it is possible to rapidly purge molecular oxygen inside the reactor to outside the reactor. Further, it is possible that supply of an olefin or an α,β-unsaturated aldehyde is stopped after the inert gas is supplied in the step of stopping the oxidation reaction.

The oxygen concentration inside the reactor attained by supplying the inert gas is preferably 10% by volume or less, more preferably 1% by volume or less, and furthermore preferably 0.01% by volume or less.

It is preferable that the inert gas to be supplied to the reactor be supplied till the step of stopping the oxidation reaction is perfectly finished. The inert gas can be supplied continuously or intermittently till the step of stopping the oxidation reaction is perfectly finished. It is preferable to supply the inert gas continuously to stop the reaction rapidly.

It is preferable to further supply a reducing agent to the reactor in the step of stopping the oxidation reaction to make surroundings of the noble metal catalyst into a reducing atmosphere. The amount of the reducing agent (g) to be supplied is preferably from V×100 to V×2,000 based on a volume of the reaction liquid in the reactor (expressed as V (L)) and more preferably from V×110 to V×1,000 in point of preventing deterioration of the noble metal catalyst by oxidation.

As the reducing agent, the one to be used at the time of the catalyst preparation mentioned above can be listed, however, an olefin or an α,β-unsaturated aldehyde is preferable. It is more preferable to use the olefin or the α,β-unsaturated aldehyde as the reducing agent, which is a raw material of the oxidation reaction to produce an α,β-unsaturated carboxylic acid because the operation of starting the reaction can be carried out stably without affecting the main reaction in the case that the reaction is restarted after the reaction has been stopped. Further, it is preferable to use a reducing agent which is liquid at a temperature and a pressure in the reactor, in particular, to use an olefin or an α,β-unsaturated aldehyde which is liquid at a temperature and a pressure in the reactor because the noble metal in a liquid phase is put into a reducing atmosphere by the reducing agent to be supplied.

The concentration of the reducing agent in the reaction liquid is not particularly limited, however, it is preferably 0.1 to 50% by mass, and more preferably 1.0 to 20% by mass because the operation of starting the reaction can be carried out stably in the case that the oxidation reaction is restarted and further because oxidation of the noble metal catalyst can be prevented without lowering the reducing ability in the reactor.

It is preferable to lower the temperature inside the reactor after or at the same time when the supply of the inert gas to the reactor has been started. Further, it is more preferable to lower the temperature inside the reactor after the supply of the inert gas to the reactor has been started and after the supply of the reducing agent to the reactor has been started.

The step of stopping the oxidation reaction is completed by returning the pressure inside the reactor to the normal pressure after the oxygen concentration and the temperature inside the reactor are sufficiently lowered. It is preferable to return the pressure inside the reactor to the normal pressure when the temperature inside the reactor becomes 50° C. or below and the oxygen concentration inside the reactor becomes 1% by volume or less.

### EXAMPLES

Hereinafter, the present invention will be explained in more detail by examples, however, the present invention is not limited to these examples.

### (Catalyst Preparation)

To 2,640 g of a mixed solution of 88% by mass n-valeric acid and 12% by mass water, 48 g of palladium acetate (manufactured by N.E. CHEMCAT Corporation) was dissolved. The resultant solution was transferred to an autoclave and 240 g of an activated carbon was added to the solution. The autoclave was shut tight and a gas phase portion of the autoclave was substituted by nitrogen while a liquid phase portion was stirred, and the resultant system was cooled till the liquid phase reaches 5 to 10° C. Then, propylene was introduced into the autoclave till the inside pressure reaches 0.5 MPaG, and stirring of the liquid phase was carried out for 1 hour at 50° C. Subsequently, the stirring was stopped, and the pressure inside the autoclave was released, and the reaction liquid was taken out. A precipitate was filtrated from the resultant reaction liquid under nitrogen flow, and a supported palladium catalyst was obtained. The loading ratio of palladium of this catalyst was 10% by mass.

### (Reactor)

A stirred tank type gas-liquid-solid catalytic reactor having inside volume of 4 L was used as a reactor. The reactor is equipped with a device which can continuously supply a gas containing molecular oxygen from a lower part of the reactor, a pressure controlling device which keeps the pressure of the gas phase inside the reactor constant, and a device which can continuously supply a liquid raw material. Further, the reactor has a structure in which the reaction liquid is drawn out while the liquid level of the liquid phase is kept constant, and the catalyst is filtrated, and the resultant filtrate can be continuously drawn out to outside of the system.

### Example 1

### First Oxidation Reaction

To the reactor, 264 g of the supported palladium catalyst and 2.5 L of 75% by mass t-butanol aqueous solution were introduced, and the reactor was pressurized by nitrogen to 4.8 MPaG. Then, 250 g of isobutylene was introduced to the reactor, and a liquid raw material prepared by adding 25 parts by mass of isobutylene to 100 parts by mass of 75% by mass t-butanol aqueous solution was continuously supplied in such a way that an average residenc time of the liquid raw material in the reactor becomes 0.9 hour. At this time, the reaction liquid was drawn out while the liquid level in the reactor was kept constant, and the catalyst was filtrated, and the resultant filtrate was continuously drawn out. Subsequently, air was continuously supplied at a rate of 620 NL/hr and, at the same time, the temperature of the liquid phase was raised to 90° C. and the reaction was started. The filtrate which had been continuously drawn out was analyzed when 91 hours had passed from the start of the reaction. The results of the reaction were: isobutylene conversion of 25.0%, selectivity to methacrolein of 50.3%, and selectivity to methacrylic acid of 33.0%. At this time, oxygen concentration in a gas drawn out from an upper space portion of the reactor was 4.3% by volume.

The analysis of the above raw materials and products were carried out using gas chromatography. Isobutylene conversion, and selectivities to methacrolein and methacrylic acid produced are defined as follows.

Isobutylene conversion (%)=(B/A)×100

Selectivity to methacrolein (%)=(C/B)×100

Selectivity to methacrylic acid (%)=(D/B)×100

In the above formulae, A represents number of moles of isobutylene supplied, B represents number of moles of isobutylene reacted, C represents number of moles of methacrolein produced, and D represents number of moles of methacrylic acid produced.

### (Step of Stopping the Oxidation Reaction)

After the foregoing oxidation reaction was finished, the supply of air was stopped, and nitrogen, the volume of which was 5.7 times (at 0° C., 1 atm) as much as the volume of a fluid dispersion in the reactor, in which the supported palladium catalyst was dispersed, was supplied at the rate of 620 NL/hr. The supply of the liquid raw material was continued for 1 hour after the supply of air was stopped, and stopped. At this time, oxygen concentration in a gas drawn out from an upper space portion of the reactor was 0.0% by volume. As such, the operational safety at the time of stopping the reaction is ensured.

### (Second Oxidation Reaction)

Subsequently, the same oxidation reaction as the first one was carried out again. The filtrate which had been continuously drawn out was analyzed when 91 hours had passed from the start of the reaction. The results of the reaction were: isobutylene conversion of 25.5%, selectivity to methacrolein of 43.5%, and selectivity to methacrylic acid of 33.8%. As such, deterioration of the supported palladium catalyst can be prevented. At this time, oxygen concentration in a gas drawn out from an upper space portion of the reactor was 5.2% by volume.

### Comparative Example 1

### First Oxidation Reaction

An oxidation reaction was carried out with the same procedure as in Example 1.

### (Step of Stopping the Oxidation Reaction)

After the foregoing oxidation reaction is finished, the supplies of air and the liquid raw material are stopped simultaneously, and the reaction is stopped. At this time, oxygen concentration in a gas drawn out from an upper space portion of the reactor exceeds 6.0% by volume and further rises. As such, the operational safety at the time of stopping the reaction is not ensured.

### (Second Oxidation Reaction)

Subsequently, the same oxidation reaction as the first one is carried out again, however, the supported palladium catalyst is deteriorated and the results of the reaction are lowered.

1. A method for producing an α,β-unsaturated carboxylic acid by an oxidation reaction in which an olefin or an α,β-unsaturated aldehyde is oxidized in a liquid phase in the presence of a noble metal catalyst in a reactor to obtain the α,β-unsaturated carboxylic acid, the method comprising the step of:
stopping the oxidation reaction by supplying an inert gas to the reactor.

2. The method for producing an α,β-unsaturated carboxylic acid according to claim 1, wherein the oxidation reaction is carried out continuously by supplying the olefin or the α,β-unsaturated aldehyde, a solvent and molecular oxygen to the reactor, and supply of the molecular oxygen is stopped before the inert gas is supplied in the step of stopping the oxidation reaction.

3. The method for producing an α,β-unsaturated carboxylic acid according to claim 2, wherein supply of the olefin or the α,β-unsaturated aldehyde is stopped after the inert gas is supplied in the step of stopping the oxidation reaction.

4. The method for producing an α,β-unsaturated carboxylic acid according to claim 2, wherein a rate of supply of the inert gas to be supplied to the reactor in the step of stopping the oxidation reaction is 1 to 100 times as much as a rate of supply of the molecular oxygen to be supplied in the oxidation reaction.

5. The method for producing an α,β-unsaturated carboxylic acid according to claim 1, wherein a total volume of the inert gas to be supplied to the reactor, at 0° C. and under 1 atm, in the step of stopping the oxidation reaction is 1 to 1,000 times as much as a volume of a reaction liquid in the reactor.

6. The method for producing an α,β-unsaturated carboxylic acid according to claim 1, wherein a total volume of the inert gas to be supplied to the reactor, at 0° C. and under 1 atm, in the step of stopping the oxidation reaction is 1 to 1,000 times as much as a volume of the reactor.

7. The method for producing an α,β-unsaturated carboxylic acid according to claim 1, wherein a reducing agent is further supplied to the reactor in the step of stopping the oxidation reaction.

8. The method for producing an α,β-unsaturated carboxylic acid according to claim 7, wherein the reducing agent is an olefin or an α,β-unsaturated aldehyde which is liquid at a temperature and a pressure in the reactor.

9. The method for producing an α,β-unsaturated carboxylic acid according to claim 7, wherein an amount of the reducing agent (g) to be supplied to the reactor in the step of stopping the oxidation reaction is, based on a volume V (L) of the reaction liquid in the reactor, from V×100 to V×2,000.
